(19) United States
(12) Patent Application Publication
(10) Pub. No.: US 2012/0065424 A1
(43) Pub. Date: Mar. 15, 2012

(54) LOW-CATALYST CARBODIIMIDE GROUPS AND/OR ISOCYANATE MIXTURES COMPRISING URETONIMINE GROUPS

(75) Inventors: Emmanouil Spyrou, Schermbeck (DE); Evelyn Albrecht, Recklinghausen (DE); Annegret Lilienthal, Dorsten (DE); Iris Brueckner, Dorsten (DE); Andrea Voecker, Ahaus (DE)

(73) Assignee: EVONIK DEGUSSA GmbH, Essen (DE)

(21) Appl. No.: 13/322,165

(22) PCT Filed: Apr. 21, 2010
(86) PCT No.: PCT/EP2010/055244

§ 371 Date: Nov. 23, 2011

(30) Foreign Application Priority Data
Jun. 26, 2009 (DE) 102009027246.1

(51) Int. Cl. C07C 265/00

(52) U.S. Cl. 560/334

(57) ABSTRACT

A low-catalyst-content isocyanate mixture containing at least one of a carbodiimide group and a uretonimine groups, obtained by: reacting at least one monomeric diisocyanate (A) in the presence of phosphorus-containing catalyst (B), to obtain a reaction mixture comprising carbodiimide, where the reaction does not take place to complete conversion of the diisocyanate (A) and 1-80% by weight of the diisocyanate (A) employed remains in the reaction mixture as excess diisocyanate; and subsequently simultaneously removing, by distillation, a fraction of the excess of diisocyanate and the catalyst (B), to obtain the isocyanate mixture and a distillate, where the isocyanate mixture has a monomeric diisocyanate (A) content of 0.5%-20% by weight, based on the monomeric diisocyanate (A) employed, and a catalyst (B) content of 0% to 20% by weight, based on the catalyst (B) employed. In addition, a process for preparing a low-catalyst-content isocyanate mixture.

The invention relates to low-catalyst-content isocyanate mixtures containing carbodiimide groups and/or uretonimine groups, to a process for the preparation, and to their use.

The carbodiimidization of isocyanates is a known process and is described in numerous patent applications. For instance, processes for preparing isocyanate mixtures containing carbodiimide and/or uretonimine groups, with the catalysts of the phospholene oxide series that are very effective for this reaction, are known from U.S. Pat. No. 2,853,473 and EP 515 933, for example.

A consequence of using the phospholene oxides as catalysts for the carbodiimidization reaction is that catalyst remaining in the product must be effectively stopped (deactivated) if storage-stable low-color isocyanate mixtures containing carbodiimide and/or uretonimine groups and having a defined NCO content are to be prepared. Otherwise, carbodiimidized isocyanate mixtures tend toward subsequent reaction and continue to give off CO₂. There are changes in the product on storage, and a build up of pressure in closed containers.

A number of specifications address the possibilities for the stopping of the carbodiimidization reaction:

Suitable deactivators for the phospholene carbodiimide catalyst are referred to in patents EP 515 933, EP 609 698, and U.S. Pat. No. 6,120,699, for example, and contain, for example, acid, acid chlorides, chloroformates and silylated acids.

EP 1 616 858 as well discloses a process for preparing organic isocyanates containing carbodiimide and/or uretonimine groups by partial carbodiimidization of isocyanate groups with catalysts of the phospholene type. In this case the carbodiimidization reaction is stopped by the addition of a silylated acid and by the further addition of a nonsilylated acid and/or an acid chloride and/or a sulfonic ester.

EP 1 616 858 thus describes the preparation of liquid, storage-stable isocyanate mixtures with low color numbers by deactivation of the phospholene catalyst.

U.S. Pat. No. 4,068,055 and U.S. Pat. No. 4,068,065 describe polymer-bound phospholene catalysts which can be removed again by filtration after the carbodiimide reaction. On the one hand, polymeric catalysts of this kind are difficult to prepare, and of poorer activity, and on the other hand it is totally impossible, or possible only with great technical cost and complexity, to remove the catalysts from carbodiimide mixtures that are solid or of high viscosity.

DE-OS-102 06 112 describes aqueous dispersions composed of polycarbodiimides. Here, residues of unreacted TMXDI are removed by distillation from a tetramethylenxylylene diisocyanate (TMXDI)/polycarbodiimide mixture after preparation; accordingly, virtually no monomeric diisocyanate remains in the end is product, and reaction then takes place with an alcohol to form a polycarbodiimidurethane containing carbodiimide groups. Removal of the catalyst is not mentioned and, as is evident from page 3, line 68, the catalyst is deactivated by blocking with acid chlorides customarily when using phospholene oxides as a catalyst for the preparation of carbodiimides. There was also no determination of the catalyst content, either in the residue or in the distillate.

The complete removal of other monomeric diisocyanates from polycarbodiimide mixtures has not hitherto been described, since either it is difficult because of excessive boiling points (e.g. dicyclohexylmethylene diisocyanate (H₁₂MDI), and/or cannot be implemented technically because of the reaction of aliphatic isocyanates with carbodiimides (uretonimine formation) (e.g., isophorone diisocyanate (IPDI), hexamethylene diisocyanate (HDI), trimethylhexamethylene diisocyanate (TMDI)). The formation of uretonimine means that a not inconsiderable fraction (generally 1%-15% by weight) of monomeric diisocyanates remains in the residue.

Practice shows that an isocyanate mixture containing carbodiimide and/or uretonimine groups, as a result of the phosphorus-containing catalyst remaining in the product, on the one hand is not storage-stable and on the other hand tends toward unwanted discoloration. State of the art, therefore, are a wide variety of costly and inconvenient efforts to deactivate the catalyst. The substances added for deactivation to isocyanate mixtures containing carbodiimide and/or uretonimine groups, however, are secondary components that are in some cases hazardous to health and expensive, and are therefore unwanted. The phosphorus-containing catalysts as well are harmful to health and in fact very expensive.

It was an object of the present invention, therefore, to provide a process for preparing storage-stable isocyanate mixtures containing carbodiimide and/or uretonimine groups that does not have the aforementioned deficiencies, and that leads to storage-stable, carbodiimide-containing products with a low color number.

Surprisingly it has been found that low-catalyst-content isocyanate mixtures containing carbodiimide groups and/or uretonimine groups can be prepared by simultaneous distillative removal of a fraction of monomeric diisocyanates and catalysts. Although the amount of monomeric diisocyanates is not lowered to the usual degree (<0.5% by weight) in this case, the fraction of the catalyst is nevertheless lowered to a maximum of 0-20% of the original concentration.

The invention provides a low-catalyst-content isocyanate mixture containing carbodiimide groups and/or uretonimine groups, prepared by reacting

### I)

A) at least one diisocyanate
in the presence of
B) phosphorus-containing catalysts for carbodiimide formation, the reaction not taking place to complete conversion of the diisocyanate and 1-80% by weight of the diisocyanate used remaining in the reaction mixture;

### II)

with subsequent simultaneous distillative removal of a part of the excess monomeric diisocyanate A) and of the phosphorus-containing catalyst B);
with the isocyanate mixture having a monomeric diisocyanate A) content of 0.5%-20% by weight, based on the diisocyanate A) employed, and a catalyst B) content of 0% to 20% by weight, based on the catalyst B) employed.

The carbodiimides content of the low-catalyst-content isocyanate mixture of the invention (residue), containing carbodiimide groups and/or uretonimine groups, is between 0.1° A) by weight and 50% by weight.

The invention also relates to a process for preparing a low-catalyst-content isocyanate mixture containing carbodiimide groups and/or uretonimine groups by partial carbodiimidization of isocyanate groups with phosphorus-containing catalysts and subsequent distillative removal of a portion of the monomeric diisocyanate employed and at the same time of the catalyst. The catalyst in this case may likewise be separated off either partially or else completely.

The low-catalyst-content isocyanate mixtures of the invention, containing carbodiimide groups and/or uretonimine groups, are particularly low-color, storage-stable mixtures. The predominant part of the phosphorus-containing catalyst removed is in the monomeric diisocyanate removed, and can be used again directly for further carbodiimidization.

The diisocyanates A) used in accordance with the invention may consist of any desired aliphatic, cycloaliphatic and/or (cyclo)aliphatic, or aromatic, diisocyanates.

Suitable aliphatic diisocyanates possess advantageously 3 to 16 carbon atoms, preferably 4 to 12 carbon atoms, in the linear or branched alkylene radical, and suitable cycloaliphatic or (cyclo)aliphatic diisocyanates possess advantageously 4 to 18 carbon atoms, preferably 6 to 15 carbon atoms, in the cycloalkylene radical. By (cyclo)aliphatic diisocyanates, the skilled person adequately understands NCO groups attached at the same time cyclically and aliphatically, as is the case with isophorone diisocyanate, for example. In contrast, cycloaliphatic diisocyanates are understood to be those which contain NCO groups only attached directly to the cycloaliphatic ring, an example being H₁₂MDI. Examples are cyclohexane diisocyanate, methylcyclohexane diisocyanate, ethylcyclohexane diisocyanate, propylcyclohexane diisocyanate, methyldiethylcyclohexane diisocyanate, propane diisocyanate, butane diisocyanate, pentane diisocyanate, hexane diisocyanate, heptane diisocyanate, octane diisocyanate, nonane diisocyanate, decane diisocyanate, undecane diisocyanate, and/or dodecane diisocyanates.

Likewise suitable are methyldiphenyl diisocyanate (MDI), 2,4- and/or 2,6-tolyl diisocyanate (TDI), 4-methylcyclohexane 1,3-diisocyanate, 2-butyl-2-ethylpentamethylene diisocyanate, 3(4)-isocyanatomethyl-1-methylcyclohexyl isocyanate, 2-isocyanatopropylcyclohexyl isocyanate, 2,4′-methylene-bis(cyclohexyl) diisocyanate, 1,4-diisocyanato-4-methylpentane.

Particularly suitable aliphatic (cyclo)aliphatic and/or cycloaliphatic diisocyanates A) are as follows: isophorone diisocyanate (IPDI), hexamethylene diisocyanate (HDI), diisocyanatodicylcohexylmethane (H₁₂MDI), 2-methylpentane diisocyanate (MPDI), 2,2,4-trimethylhexamethylene diisocyanate/2,4,4-trimethylhexamethylene diisocyanate (TMDI), and norbornane diisocyanate (NBDI). Especially preferred are IPDI, HDI, and H₁₂MDI.

It is of course also possible to use mixtures of the diisocyanates.

The isocyanate mixture of the invention containing carbodiimide groups and/or uretonimine groups is prepared in the presence of the high-activity catalysts B). An exhaustive description of suitable catalysts and preparation methods is found in, for example, Houben-Weyl, Methoden der organischen Chemie, Volume E4, carbonic acid derivatives, Georg-Thieme-Verlag, Stuttgart, 1983, pp. 897 to 900 and 910, and also in Chemical Reviews, Volume 67, Number 2, 1967, pp. 107-113, or in Angew. Chem., 1962, No. 21, 801-806. Carbodiimidization catalysts are also described in U.S. Pat. No. 2,941,966, U.S. Pat. No. 2,853,518, U.S. Pat. No. 2,853,473 or DE 35 12 918. Preferred catalysts are phospholenes and phospholanes and also their oxides and sulfides, more preferably of the phospholene oxide type. Examples of catalysts frequently employed are 1-methyl-2-phospholene 1-oxide, 1-methyl-3-phospholene 1-oxide, 3-methyl-1-phenyl-3-phospholene 1-oxide, and 3-methyl-1-phenyl-2-phospholene 1-oxide, and also the corresponding phospholane types. It is preferred to use 3-methyl-1-phenyl-2-phospholene 1-oxide. Likewise suitable are phosphine oxides. The amount of catalyst, based on the diisocyanate A), is 0.1% to 3% by weight, preferably 0.5%-1.5% by weight.

The isocyanate mixture of the invention containing carbodiimide groups and/or uretonimine groups is preferably prepared, in accordance with step I), by a process where at least one of the isocyanates stated under A) is reacted, with addition of at least one of the catalysts listed under B), by heating to temperatures of 30-200° C., with elimination of carbon dioxide. The temperature is preferably 80-200° C., the duration preferably between 30 min and 24 h. In this case, depending on catalyst content, temperature and time, smaller or larger amounts of monomeric diisocyanate remain in the reaction mixture, preferably from 1% to 80% by weight, based on the amount A) employed.

The distillative simultaneous removal II) of a fraction of monomeric diisocyanate and of phosphorus-containing catalyst takes place in suitable distillation assemblies, in short-path evaporators, thin-film evaporators or falling-film evaporators, for example. The temperature, depending on the boiling point of the diisocyanate employed, is 100 to 240° C., preferably 130 to 200° C. In this context it may be advantageous for the mixture that is to be distilled to be preheated even before the actual distillation to a temperature between 100 and 200° C., preferably between 120 and 160° C. The pressure is maintained between 0.001 mbar and 50 mbar, preferably between 0.01 and 10 mbar. The monomeric diisocyanate content of the residue, in other words of the low-catalyst-content isocyanate mixture of the invention containing carbodiimide groups and/or uretonimine groups, is, after distillation, 0.5%-20% by weight, preferably 3% to 10% by weight. After the distillation, the catalyst used is present at about 80%-100% in the distillate, and at about 0%-20% in the residue, based on the amount of catalyst employed. It is preferably present at 95%-99% in the distillate and at 1%-5% in the residue. Smaller amounts of catalysts (0%-10%, based on the overall amount employed) may also remain in the cold trap or on the distillation apparatus. The carbodiimides content of the residue is between 0.1% by weight and 50% by weight.

The invention also provides a process for preparing a low-catalyst-content isocyanate mixture containing carbodiimide groups and/or uretonimine groups by reacting

### I)

A) at least one diisocyanate
in the presence of
B) phosphorus-containing catalysts for carbodiimide formation, the reaction not taking place to complete conversion of the diisocyanate and 1-80% by weight of the diisocyanate used remaining in the reaction mixture;

### II)

with subsequent simultaneous distillative removal of a part of the excess monomeric diisocyanate A) and of the phosphorus-containing catalyst B);
with the isocyanate mixture having a monomeric diisocyanate A) content of 0.5%-20% by weight, based on the diisocyanate A) employed, and a catalyst B) content of 0% to 20% by weight, based on the catalyst B) employed.

Carbodiimide-containing compounds find use in the coatings and adhesives industry and also, generally, in the plastics industry as stabilizers, and/or as crosslinkers.

### EXAMPLES

### General Determination Methods:

The NCO content is determined titrimetrically by reaction of the NCO groups with dibutylamine and subsequent back-titration of the excess dibutylamine with hydrochloric acid. The hot value is determined after 30-minute heating of the sample at 180° C. and sudden cooling. The carbodiimide content is determined after a two-hour boiling with butanol, with copper(I) chloride catalysis, and subsequent reaction with dibutylamine, followed by back-titration of the excess dibutylamine with hydrochloric acid.

1a) Preparation of the Carbodiimides from IPDI

1581.4 g of IPDI (Evonik-Degussa) are reacted fractionally to give the carbodiimide with 18.6 g of 3-methyl-1-phenyl-2-phospholene 1-oxide (Alfa Aesar) under a permanent N₂ stream at 110° C. in 3.5 h with elimination of CO₂. Some of the carbodiimide reacts further in a reversible reaction with free isocyanate to give uretonimine. The data found for the reaction product was as follows:

NCO content cold: 27.66% by weight—NCO content hot: 30.46% by weight—Carbodiimide content: 3.55% by weight—Hazen color: 96, Viscosity 23° C.: 691 mPas

1b) Removal of the Phosphorus-Containing Catalysts by Distillation, and Preparation of the Low-Catalyst-Content Isocyanate Mixture Containing Carbodiimide Groups and/or Uretonimine Groups

539 g of the carbodiimide prepared from IPDI under 1a) is subjected to a short-path distillation (KDL4, UIC) at approximately 200 g/h at 155° C. and 0.2 mbar. 145.6 g are collected as a residue. This residue contains 8.2% by weight of monomeric IPDI and 110 ppm of phosphorus. This corresponds to approximately 1.4% of the catalyst employed.

The distillate (375.4 g) contains 2800 ppm of phosphorus, corresponding to 95.5% of the catalyst employed. NCO content cold: 10.95% by weight—NCO content hot: 14.11% by weight—carbodiimide content: 10.4% by weight—Hazen color (30% in toluene): 115—m.p. 64° C.

2a) Further Preparation of Carbodiimide from IPDI Distillate from 1b)

350 g of IPDI distillate from 1b) are diluted with 221 g of IPDI (Evonik-Degussa). The reaction takes place fractionally to give carbodiimide under a permanent N₂ stream at 110° C. over the course of 3 h with CO₂ elimination. The data determined for the reaction product are as follows:

NCO content cold: 28.24%—NCO content hot: 30.87%—carbodiimide content: 3.54%—Hazen color: 91—Gardner color: 0.2—viscosity 23° C.: 629 mPas

2b) Further Removal of the Phosphorus-Containing Catalyst by Distillation, and Preparation of the Low-Catalyst-Content Isocyanate Mixture Containing Carbodiimide Groups and/or Uretonimine Groups

400.6 g of the carbodiimide prepared from IPDI under 2a) is subjected to a short-path distillation (KDL4, UIC) at approximately 200 g/h at 155° C. and 0.2 mbar. 85.2 g are collected as a residue. This residue contains 8.4% of monomeric IPDI and 180 ppm of phosphorus. This corresponds to approximately 2.1% of the catalyst employed.

The distillate (297.5 g) contains 2500 ppm of phosphorus, corresponding to 95.6% of the catalyst employed. NCO content cold: 9.85% by weight—NCO content hot: 12.60% by weight—carbodiimide content: 10.8% by weight—Hazen color (30% in toluene): 107—m.p. 78° C.

From these examples it can be seen that the major fraction of the phosphorus-containing catalyst remains in the distillate. It can be used again without restrictions, whereas the residue is largely of low catalyst content.

### Storage Stability

For the determination of the storage stability, the products 1a) (before distillation), 1b) (after distillation) and 1b)+1% by weight of catalyst (3-methyl-1-phenyl-2-phospholene 1-oxide), were stored at 50° C. for 7 days and the NCO content (hot) found was compared with the initial NCO content (hot).

| Product | la*) | 1b) | 1b) + 1% catalyst* |
|---|---|---|---|
| NCO content (hot) [%] | 30.6 | 14.1 | 14.0 |
| NCO content (hot) [%] after 7 d at 50° C. | 25.7 | 13.9 | 12.5 |
| Decrease relative to initial value | 16% | 1% | 11% |

*noninventive comparative experiments

In these examples it can be seen that only the low-catalyst-content product 1b) is storage-stable (decrease in the hot NCO number after 7 days at 50° C. is smaller than 5%). The two comparison products 1a) and 1b)+1% catalyst show a subsequent reaction after storage at 50° C., formation of further carbodiimide with elimination of carbon dioxide, resulting in a decrease in the NCO number of more than 5% after a week.

1. A low-catalyst-content isocyanate mixture comprising at least one selected from the group consisting of a carbodiimide group and a uretonimine group, obtained by a process comprising:
(I) reacting at least one monomeric diisocyanate (A) in the presence of a phosphorus-comprising catalyst (B), to obtain a reaction mixture comprising carbodiimide, wherein the reacting does not take place to complete conversion of the monomeric diisocyanate (A) and 1-80% by weight of the monomeric diisocyanate (A) employed remains in the reaction mixture as excess diisocyanate; and subsequently
(II) simultaneously removing by distillation, a fraction of the excess diisocyanate and a fraction of the catalyst (B) to obtain the isocyanate mixture and a distillate,
wherein the isocyanate mixture comprises the monomeric diisocyanate (A) in a content of 0.5%-20% by weight, based on the monomeric diisocyanate (A) employed, and a catalyst (B) content of 0% to 20% by weight, based on the catalyst (B) employed.

2. The isocyanate mixture of claim 1, wherein the monomeric diisocyanate (A) is at least one selected from the group consisting of isophorone diisocyanate (IPDI), hexamethylene diisocyanate (HDI), diisocyanatodicylcohexylmethane (H₁₂MDI), 2-methylpentane diisocyanate (MPDI), 2,2,4-trimethylhexamethylene diisocyanate (2,2,4-TMDI), 2,4,4-trimethylhexamethylene diisocyanate (2,4,4-TMDI), and norbornane diisocyanate (NBDI).

3. The isocyanate mixture of claim 1, wherein the diisocyanate is at least one selected from the group consisting of IPDI, HDI, and H₁₂MDI.

4. The isocyanate mixture of claim 1, wherein the catalyst (B) is at least one selected from the group consisting of phospholene, a phospholane, a phospholene oxide, a phospholane oxide, a phospholene sulfide, and a phospholane sulfide.

5. The isocyanate mixture of claim 1, wherein the catalyst (B) is at least one selected from the group consisting of 1-methyl-2-phospholene 1-oxide, 1-methyl-3-phospholene 1-oxide, 3-methyl-1-phenyl-3-phospholene 1-oxide, 3-methyl-1-phenyl-2-phospholene 1-oxide, 1-methylphospholane 1-oxide, and 3-methyl-1-phenylphospholane 1-oxide.

6. The isocyanate mixture of claim 1, wherein an amount of the catalyst (B), based on the monomeric diisocyanate (A), is 0.1% to 3% by weight.

7. The isocyanate mixture of claim 1, further comprising, during the reacting:
heating to temperatures in a range of 30-200° C., and eliminating carbon dioxide.

8. The isocyanate mixture of claim 1, wherein the removing takes place in a short-path evaporator, a thin-film evaporator, or a falling-film evaporator.

9. The isocyanate mixture of claim 8, wherein the temperature, during the removing, is in a range of 100 to 240° C.

10. The isocyanate mixture claim 8, further comprising, before the removing, preheating the reaction mixture to a temperature between 100 and 200° C.

11. The isocyanate mixture of claim 8, wherein the removing, by distillation, is carried out at a pressure between 0.001 mbar and 50 mbar.

12. The isocyanate mixture of claim 1, further comprising, after the removing (II),
further reacting the fraction of catalyst (B) which is present in the excess diisocyanate from the removing and additional monomeric diisocyanate.

13. The isocyanate mixture of claim 1, wherein the isocyanate mixture comprises a monomeric diisocyanate (A) content of 3% to 10% by weight, based on the monomeric diisocyanate (A) employed, and a catalyst (B) content of 1% to 5% by weight, based on the catalyst (B) employed.

14. The isocyanate mixture of claim 1, wherein a carbodiimide content of the low-catalyst-content isocyanate mixture is between 0.1% by weight and 50% by weight.

15. A process for preparing a low-catalyst-content isocyanate mixture comprising at least one selected from the group consisting of a carbodiimide group and a uretonimine group, the processing comprising:
partially carbodiimidizing at least one monomeric isocyanate group and a phosphorus-comprising catalyst; and subsequently
simultaneously removing, by distillation, a fraction of excess monomeric diisocyanate employed and the catalyst.

16. A process for preparing a low-catalyst-content isocyanate mixture comprising at least one selected from the group consisting of a carbodiimide group and a uretonimine group, the process comprising:
(I) reacting at least one monomeric diisocyanate (A) in the presence of a phosphorus-comprising catalyst (B), to obtain carbodiimide, wherein the reacting does not take place to complete conversion of the monomeric diisocyanate (A) and 1-80% by weight of the monomeric diisocyanate (A) remains in the reaction mixture as excess diisocyanate; and subsequently
(II) simultaneously removing, by distillation, a fraction of the excess diisocyanate and the phosphorus-comprising catalyst (B), to obtain the low-catalyst-content isocyanate mixture,
wherein the isocyanate mixture comprises the monomeric diisocyanate (A) in a content of 0.5%-20% by weight, based on the monomeric diisocyanate (A) employed, and a catalyst (B) content of 0% to 20% by weight, based on the catalyst (B) employed.

17. The process of claim 15, wherein the low-catalyst-content isocyanate mixture has a carbodiimide content between 0.1% by weight and 50% by weight.

18. The isocyanate mixture of claim 1, wherein an amount of the catalyst (B), based on the monomeric diisocyanate (A), is 0.5% to 1.5% by weight.

19. The isocyanate mixture of claim 8, wherein the temperature, during the removing, is in a range of 130 to 200° C.

20. The isocyanate mixture claim 8, further comprising, before the removing, preheating the reaction mixture to a temperature between 120 and 160° C.
